(19) United States
(12) Patent Application Publication
(10) Pub. No.: US 2026/0276800 A1
(43) Pub. Date: Sep. 17, 2026

(54) METHOD FOR FABRICATING AN ULTRASONIC PROBE AND CORRESPONDING ULTRASONIC PROBE

(71) Applicant: VERMON, TOURS (FR)

(72) Inventor: Emmanuel MONTAUBAN, TOURS (FR)

(21) Appl. No.: 19/557,889

(22) Filed: Mar. 5, 2026

(30) Foreign Application Priority Data
Mar. 11, 2025 (FR) 25/02406

(51) Int. Cl. G01S 7/52; B06B 1/06; H10N 30/09

(52) U.S. Cl. CPC G01S 7/52079; B06B 1/0644; H10N 30/09

(57) ABSTRACT

The present description concerns a method of manufacturing an ultrasonic probe comprising:—the forming of a dielectric layer on an edge of an acoustic stack comprising a piezoelectric layer located on a first portion of an interconnection substrate, said first portion being on an acoustic attenuation layer and being continued by a second portion folded over an edge of the acoustic attenuation layer;—the forming of a conductive grounding structure on transducer elements formed in the acoustic stack and separated by notches in the acoustic stack across its thickness all the way to the interconnection substrate; said conductive grounding structure extending over the dielectric layer and being coupled to an electrical contact pad of the interconnection substrate; and—the forming of a protective layer to cover and surround the acoustic stack and the conductive grounding structure along the height of the notches.

### CROSS-REFERENCE TO RELATED APPLICATIONS

This application claims the benefit of and priority to French patent application number FR 25/02406, filed on Mar. 11, 2025, entitled “ELEMENT DE BORNIER POUR LA CONNEXION DE PLUSIEURS CONDUCTEURS ELECTRIQUES A L'AIDE DE RESSORTS LAMELLAIRES, ” the entire contents of which is hereby incorporated herein by reference.

### TECHNICAL FIELD

The present disclosure generally concerns ultrasonic probes comprising an array of ultrasonic transducer elements. The present description particularly concerns ultrasonic probes, the transducer elements of which are piezoelectric transducer elements. The present disclosure particularly concerns the manufacturing of such ultrasonic probes.

### PRIOR ART

Certain ultrasonic probes comprise an array of transducer elements arranged next to one another in a linear arrangement or in a matrix arrangement.

The transducer elements of the array are preferably individually addressed. An example of an ultrasonic probe comprises an array of transducer elements, the signals of which can be phase-shifted with respect to each other, or “phased array”.

When the transducer elements are piezoelectric transducer elements, each transducer element typically comprises a layer of piezoelectric material, or piezoelectric layer, and front and rear electrodes (or upper and lower electrodes) adapted to applying to the transducer element an electrical excitation signal and/or to recover from the transducer element a signal to be converted into an electrical signal. One of the front and rear electrodes may be connected to ground, and the other may be dedicated to the signal.

These ultrasonic probes can be used for 2D or even 3D imaging, particularly in medical imaging applications, in particular for external medical imaging, that is, with no intrusion into a body.

It would be desirable to have an ultrasonic probe at least partially overcoming some of the disadvantages of known ultrasonic probes.

It would be desirable to have a method of manufacturing such an ultrasonic probe, this method at least partially overcoming some of the disadvantages of known manufacturing methods.

### SUMMARY OF THE INVENTION

An embodiment overcomes all or part of the disadvantages of known methods of manufacturing ultrasonic probes and of the obtained ultrasonic probes.

An embodiment provides a method of manufacturing an ultrasonic probe, the method comprising:
the forming of a dielectric layer on at least one edge of an acoustic stack comprising at least one piezoelectric layer located on a first portion of an interconnection substrate, said first portion of said interconnection substrate being on an acoustic attenuation layer and being continued by a second portion of the interconnection substrate folded over an edge of the acoustic attenuation layer;
the forming of a conductive grounding structure on transducer elements formed in the acoustic stack and separated by notches resulting from the cutting of the acoustic stack across its entire thickness, said cutting extending at least all the way to the interconnection substrate; said conductive grounding structure extending over the dielectric layer and being coupled to at least one electrical contact pad of the interconnection substrate;
the forming of a protective layer to cover and surround the acoustic stack and the conductive grounding structure at least along the height of the notches.

According to an embodiment, the cutting extends into the acoustic attenuation layer and includes the cutting of at least the first portion of the interconnection substrate.

According to an embodiment, the dielectric layer is configured to protect the at least one edge of the acoustic stack intended to be covered by the conductive grounding structure. For example, an edge of the acoustic stack intended to be covered by the conductive grounding structure is aligned with the edge of the acoustic attenuation layer over which the second portion of the interconnection substrate is folded.

According to an embodiment, the forming of the conductive grounding structure comprises:
the forming of a metal layer on the dielectric layer, on top of and around the acoustic stack at least until it contacts the at least one pad; and
the cutting of the acoustic stack into the transducer elements separated from one another by the notches, said cutting further including the cutting of the metal layer.

According to an embodiment, the forming of the conductive grounding structure comprises:
the cutting f the acoustic stack into the transducer elements separated from one another by the notches; and then
the forming of a network of metal traces comprising first traces on each of the transducer elements, each first trace being continued by a second trace extending over the dielectric layer and coupled to the at least one pad.

According to an embodiment, the second traces are connected to a third trace connected to the at least one pad.

According to an embodiment, the second traces are directly connected to the at least one pad.

According to an embodiment, the method further comprises the forming of an electromagnetic shielding layer covering the edges of the acoustic attenuation layer along all or part of its height, as well as at least a portion of the interconnection substrate, the electromagnetic shielding layer being a conductive layer, for example in the form of a copper adhesive.

According to an embodiment, the electromagnetic shielding layer also covers the at least one pad.

According to an embodiment, the method further comprises the forming of a layer covering and surrounding the protective layer at least along the height of the notches so as to form a lens, the layer being, for example, a silicone layer.

According to an embodiment, the cutting of the acoustic stack includes the cutting of a first metal layer arranged above the piezoelectric layer, for example on the acoustic stack, and of a second metal layer arranged under the piezoelectric layer, for example between the acoustic stack and the acoustic attenuation layer, so as to respectively form first electrodes and second electrodes of the transducer elements, the first electrodes being coupled to the at least one pad through the conductive grounding structure.

An embodiment provides an ultrasonic probe comprising:
an interconnection substrate comprising at least a first portion and a second portion coupled to the first portion;
an acoustic stack comprising at least one piezoelectric layer located on the first portion of the interconnection substrate;
a dielectric layer on at least one edge of the acoustic stack;
an acoustic attenuation layer, the first portion of the interconnection substrate being between said acoustic attenuation layer and the piezoelectric layer, and the second portion of the interconnection substrate being folded over an edge of said acoustic attenuation layer; the acoustic stack being divided into a plurality of transducer elements separated by notches extending through said acoustic stack across its entire thickness, said notches extending at least all the way to the interconnection substrate;
a conductive grounding structure coupled to each of the transducer elements, the conductive grounding structure extending over the dielectric layer and being coupled to at least one electrical contact pad of the interconnection substrate; and
a protective layer covering and surrounding the acoustic stack and the conductive grounding structure at least along the height of the notches.

According to an embodiment, the notches extend through at least the first portion of the interconnection substrate all the way into the acoustic attenuation layer.

According to an embodiment, the ultrasonic probe further comprises:
an electromagnetic shielding layer covering the edges of the acoustic attenuation layer along all or part of its height, as well as at least a portion of the interconnection substrate; and/or
a layer forming a lens covering and surrounding the protective layer at least along the height of the notches.

The following embodiments may apply to the manufacturing method and/or to the ultrasonic probe.

According to an embodiment, the protective layer is a polymer film, for example an adhesive polymer film.

According to an embodiment, the protective layer is metallized at least on its surface which faces the conductive grounding structure.

According to an embodiment, the electrical contact pad is positioned on the second portion of the interconnection substrate.

According to an embodiment, the notches are filled with air.

According to an embodiment, the acoustic stack comprises at least one impedance matching layer on the piezoelectric layer.

### BRIEF DESCRIPTION OF THE DRAWINGS

The foregoing features and advantages, as well as others, will be described in detail in the following non-limiting description of specific embodiments with reference to the accompanying drawings, in which:

FIG. 1A is a side view showing details of an example of an ultrasonic probe;

FIG. 1B is a front view of the ultrasonic probe of FIG. 1A;

FIG. 2A, FIG. 2B, FIG. 2C, FIG. 2D, FIG. 2E, FIG. 2F, and FIG. 2G are side and front views illustrating successive steps of a manufacturing method according to an embodiment;

FIG. 3 shows an interconnection substrate of an ultrasonic probe according to an embodiment;

FIG. 4A, FIG. 4B, FIG. 4C, FIG. 4D, FIG. 4E, and FIG. 4F are side and front views illustrating successive steps of a manufacturing method according to another embodiment;

FIG. 5A shows, in side, front, and top views, a variant of the manufacturing method of FIGS. 4A to 4F; and

FIG. 5B and FIG. 5C show an interconnection substrate of an ultrasonic probe according to another embodiment.

### DESCRIPTION OF EMBODIMENTS

Like features have been designated by like references in the various drawings. In particular, the structural and/or functional features that are common among the various embodiments may have the same references and may have identical structural, dimensional and material properties.

For the sake of clarity, only those steps and elements that are useful for understanding the described embodiments have been shown and have been described in detail. In particular, the forming of the transducer elements of the described ultrasonic probes is not detailed, the described embodiments being compatible with all or most known ultrasonic transducer structures. Further, the forming of the circuits for controlling the transducer elements is not detailed, the described embodiments being compatible with usual ultrasonic transducer control circuits, or the implementation of the control circuits being within the abilities of those skilled in the art based on the indications of the present disclosure.

Unless otherwise specified, when reference is made to two elements being connected to each other, this means directly connected without any intermediate elements other than conductors, and when reference is made to two elements being coupled to each other, this means that these two elements may be connected or may be connected through one or more other elements.

In the following description, where reference is made to absolute position qualifiers, such as the terms “front”, “back”, “top”, “bottom”, “left”, “right”, etc., or relative position qualifiers, such as the terms “top”, “bottom”, “upper”, “lower”, etc., or orientation qualifiers, such as “horizontal”, “vertical”, etc., reference is made unless otherwise specified to the orientation of the drawings.

In the following description, when reference is made to the term “lateral” or “longitudinal”, reference is made to the X direction marked in the drawings, and when reference is made to the term “transverse”, reference is made to the Y direction, which is perpendicular to the X direction. When reference is made to the term “vertical”, reference is made to the Z direction marked in the drawings, which is perpendicular to the X and Y directions.

Unless specified otherwise, the expressions “about”, “approximately”, “substantially”, and “in the order of” signify plus or minus 10% or 10°, preferably of plus or minus 5% or 5°.

In the following description, when reference is made to conductive elements, reference is made, unless otherwise specified, to electrically-conductive elements. Similarly, when reference is made to insulating elements, reference is made, unless otherwise specified, to electrically-insulating elements.

In the following description, when reference is made to a pad, a track, a trace, or a connector, reference is made, unless otherwise specified, respectively to an electrical connection pad, an electrical connection trace, an electrical connection trace, or an electrical connector.

In the following description, when reference is made to a probe, reference is made, unless otherwise specified, to an ultrasonic probe comprising an array of transducer elements. Further, when reference is made to transducer elements, or transducers, reference is made, unless otherwise specified, to piezoelectric transducer elements.

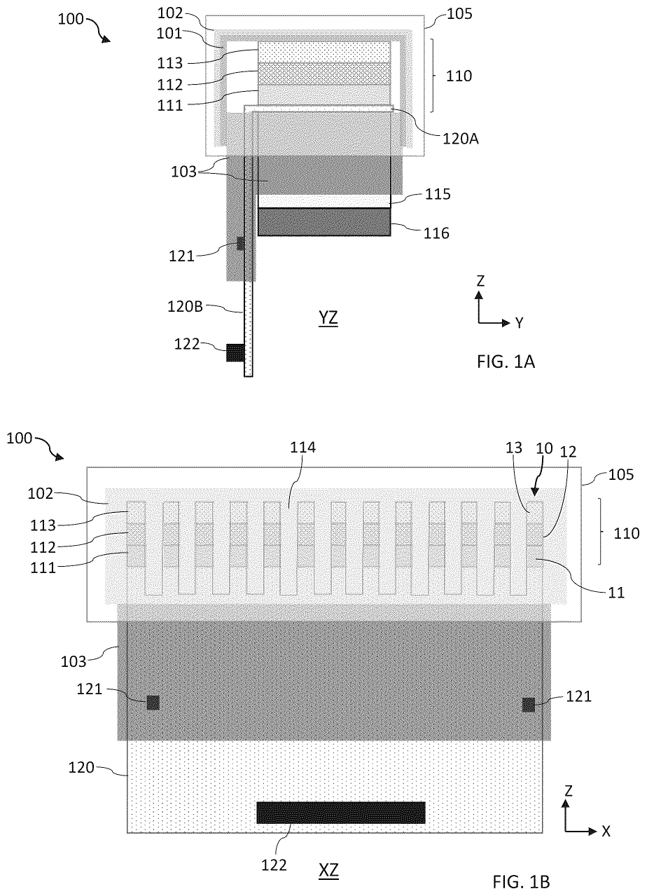

FIG. 1A is a side view showing details of an example of ultrasonic probe 100. FIG. 1B is a front view of the ultrasonic probe 100 of FIG. 1A. The side view is shown in a YZ plane (transverse plane). The front view is shown in an XZ plane (longitudinal or lateral plane).

Ultrasonic probe 100 comprises a piezoelectric layer 111.

Ultrasonic probe 100 further comprises one or more acoustic impedance matching layers, or slides, on the upper surface (front surface) of piezoelectric layer 111 intended to be oriented toward a region to be imaged. In the example of FIGS. 1A and 1B, two impedance matching layers 112 and 113 are shown: one impedance matching layer 112 on piezoelectric layer 111 and one impedance matching layer 113 on impedance matching layer 112.

The stack of layers 111, 112, and 113 can be referred to as an acoustic stack 110.

The upper and lower surfaces of acoustic stack 110 are metallized, that is, acoustic stack 110 is covered on each of its upper and lower surfaces with a metal layer: a metal layer on the upper surface (upper metal layer) and another metal layer on the lower surface (lower metal layer). Impedance matching layers 112 and 113 are then preferably at least partially conductive.

Ultrasonic probe 100 further comprises an acoustic attenuation, layer 115, or “backing”, under the lower surface (rear surface) of piezoelectric layer 111, as well as a mechanical support, or stiffener, layer 116 under acoustic attenuation layer 115.

Ultrasonic probe 100 comprises an interconnection substrate 120.

Interconnection substrate 120 comprises conductive interconnection elements (not shown in FIGS. 1A and 1B), generally in the form of interconnection tracks, or tracks, formed inside and/or on top of a dielectric support, generally flexible. The tracks are generally metallic, for example made of copper. The dielectric support is, for example, made of a polymer material, such as polyimide.

Interconnection substrate 120 is, for example, a flexible printed circuit.

Interconnection substrate 120 comprises a substantially horizontal portion 120A located between piezoelectric layer 111 and backing layer 115. The lower metal layer is in contact with interconnection substrate 120.

Interconnection substrate 120 further comprises a portion 120B coupled to portion 120A and folded over an edge, or flank, of backing layer 115 and of stiffener 116. Portion 120B may be substantially vertical. Portion 120B may extend vertically beyond stiffener 116.

Ultrasonic probe 100 further comprises an electromagnetic shielding layer 103, which is a conductive layer, for example in the form of a copper adhesive, covering the flanks of backing layer 115 along all or part of its height, as well as at least a portion of interconnection substrate 120. Electromagnetic shielding layer 103 covers, in particular, electrical contact pads 121 of interconnection substrate 120, thereby ensuring electrical continuity with these pads 121.

Acoustic stack 110 is cut along a plurality of transverse planes YZ into a plurality of transducer elements 10 separated from one another by notches 114, or kerfs. This cutting includes the cutting of each of the lower and upper metal layers into a plurality of lower and upper electrodes, respectively. Notches 114 extend at least all the way to the lower metal layer, so as to electrically isolate the lower electrodes of each £ transducer element 10 (described hereafter).

Piezoelectric layer 111 is thus divided into a plurality of piezoelectric elements 11. Acoustic impedance matching layer 112 is divided into a plurality of elements 12, and acoustic impedance matching layer 113 is divided into a plurality of elements 13. Each stack of elements 11, 12, and 13 forms a transducer element 10.

The lower electrodes are in contact with tracks of interconnection substrate 120 and form signal electrodes of the different transducer elements 10. The upper electrodes form ground electrodes of transducer elements 10.

Kerfs 114 are preferably left filled with air and are protected by a stack of layers 101 and 102 which cover and surround acoustic stack 110 at least along the height of kerfs 114. Layer 101 is a conductive film, preferably adhesive, for example an Arclad® brand adhesive conductive film. Layer 102 is a polymer film, for example made of polyethylene, which covers conductive film 101. Polymer film 102 is preferably metallized at least on its lower surface, which is in contact with conductive film 101.

Conductive film 101 has an electromagnetic shielding function.

In addition, conductive film 101 comes into contact with electromagnetic shielding layer 103 and is thus coupled to pads 121 and to interconnection substrate 120. It thus also has a grounding function for transducer elements 10 via the upper electrodes of these transducer elements.

Ultrasonic probe 100 further comprises a protective layer 105 at least on the upper surface (front surface) of acoustic stack 110, in particular on impedance matching layers 112, 113. This protective layer 105 is intended to be in contact with the region to be imaged. Protective layer 105 is made of a material allowing the propagation of ultrasonic waves, for example a biocompatible material, for example, silicone. The upper surface of protective layer 105 may be flat or form an acoustic lens, for example, be convex, capable of focusing ultrasonic waves. This protective layer 105 may be referred in the rest of the disclosure to as a silicone layer or a silicone lens, without this being restrictive of its shape and of its material. In the shown example, silicone layer 105 covers and surrounds the stack of layers 101 and 102 at least along the height of kerfs 114, although this is not limiting.

Silicone layer 105 may be formed by molding. Layers 101 and 102 advantageously protect air-filled kerfs 114 during this molding process.

Layers 102, 103, and 105 have been shown in transparency to visualize the elements which are located underneath.

Interconnection substrate 120 further comprises a connector 122 which is not covered by layers 101, 102, 103, and 105. Connector 122 may be used to couple each of the tracks of interconnection substrate 120 to another electrical connection element, for example to another interconnection substrate, a rigid or flexible printed circuit, a cable, a cable harness, or more generally any element enabling to couple the electrodes of transducer elements 10 to electronic circuits external or internal to ultrasonic probe 100.

A disadvantage of this type of ultrasonic probe 100 is that, when the lens is formed by molding of silicone layer 105, a silicone retraction force during post-polymerization shrinkage may cause the detaching of conductive film 101 and of polymer film 102, creating contact defects with acoustic stack 110 and/or on electromagnetic shielding layer 103, which negatively affects the grounding of ultrasonic probe 100, thereby decreasing its performance.

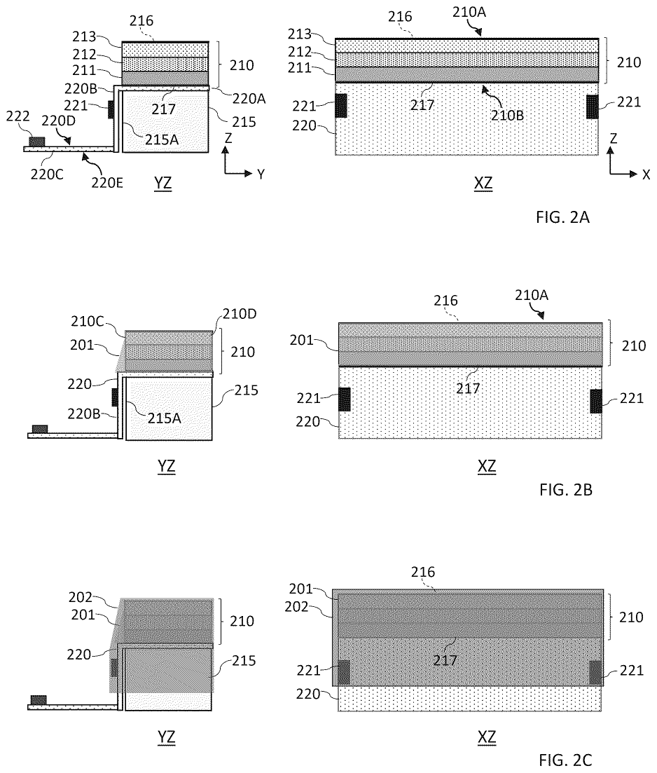

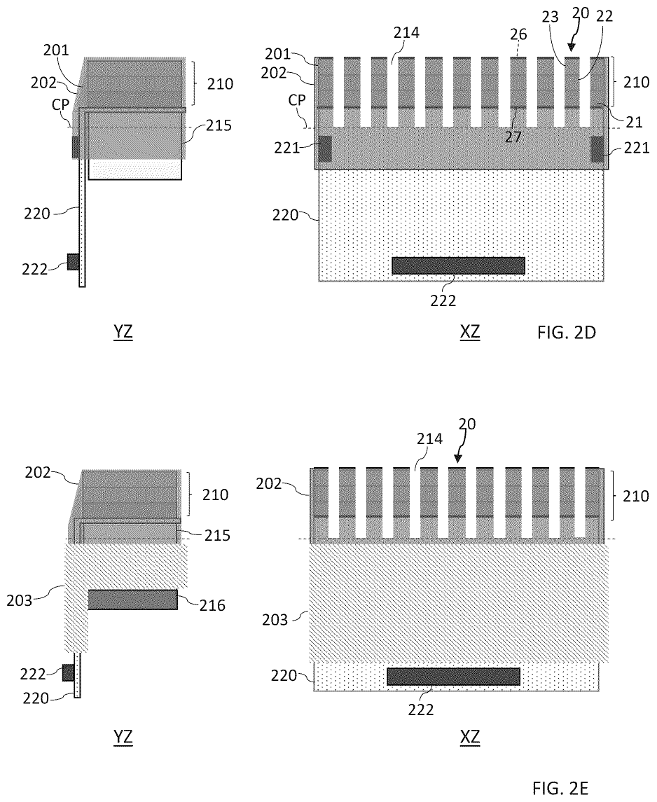

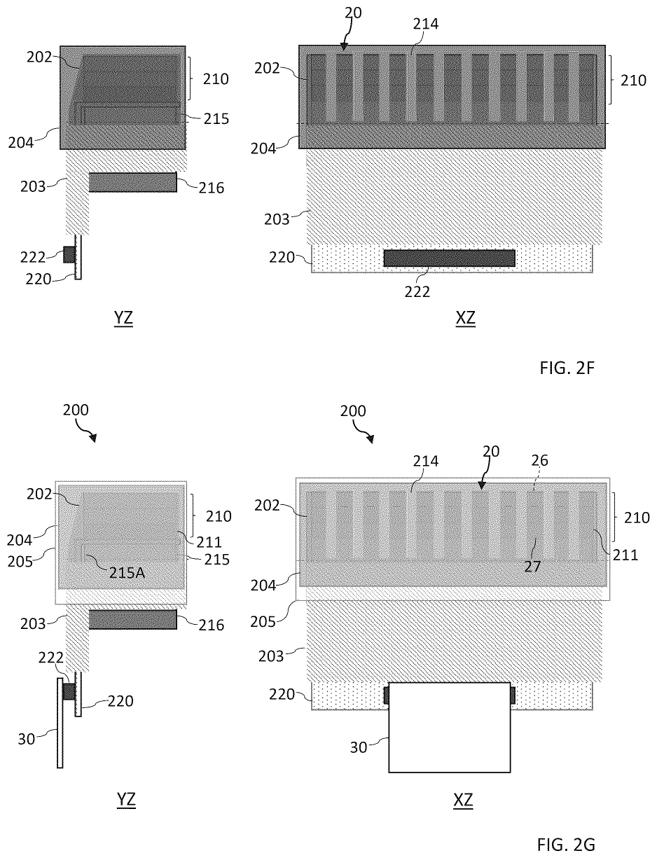

FIG. 2A, FIG. 2B, FIG. 2C, FIG. 2D, FIG. 2E, FIG. 2F, and FIG. 2G are side and front views illustrating successive steps of a manufacturing method according to an embodiment. The side views are shown in a YZ plane (transverse plane). The front views are shown in an XZ plane (longitudinal or lateral plane).

FIG. 2A shows an initial structure comprising an acoustic stack 210, similar to the acoustic stack 110 of FIGS. 1A and 1B, comprising a piezoelectric layer 211, and one or more acoustic impedance matching layers, or slides, on the upper surface (front surface) of the piezoelectric layer 211 intended to be oriented toward a region to be imaged.

In the example of FIG. 2A, two acoustic impedance matching layers 212 and 213 have been shown: an acoustic impedance matching layer 212 on piezoelectric layer 211 and another acoustic impedance matching layer 213 on acoustic impedance matching layer 212. As a variant, there could be a single impedance matching layer (for example, no impedance matching layer 213), or also more than two impedance matching layers.

At least the lower surface 210B of acoustic stack 210 is metallized, that is, acoustic stack 210 is covered at least on its lower surface 210B with a lower metal layer 217.

Impedance matching layers 212 and 213 are, for example, at least partially conductive. An upper metal layer 216 may then be provided on impedance matching layer 213.

According to a variant, impedance matching layer 212 could be conductive, and impedance matching layer 213 could be non-conductive, in which case an upper metal layer 216 could be provided between the two impedance matching layers 212 and 213.

According to another variant, the two impedance matching layers 212 and 213 could be non-conductive, and an upper metal layer 216 could then be provided between piezoelectric layer 211 and impedance matching layer 212, for example on the upper surface of piezoelectric layer 211.

The following steps of the method described hereafter in relation with FIGS. 2B to 2D are then adapted so that the grounding can be achieved on upper metal layer 216, whatever its location.

In particular, it is preferably provided for the dielectric insulation layer 201 described hereafter in relation with FIG. 2B to extend vertically from interconnection substrate 220 to the lower surface of upper metal layer 216. Then, it is preferably provided for the metallization step described in relation with FIG. 2C to form a metal layer 202 which comes into contact with upper metal layer 216, and which can come into contact with the non-insulated matching layer(s).

An interconnection substrate 220 is assembled under the lower surface 210B of acoustic stack 210, under piezoelectric layer 211, preferably in contact with lower metal layer 217. Interconnection substrate 220 also extends above an acoustic attenuation layer 215, or backing layer, and over an edge 215A, or flank 215A, of this acoustic attenuation layer 215.

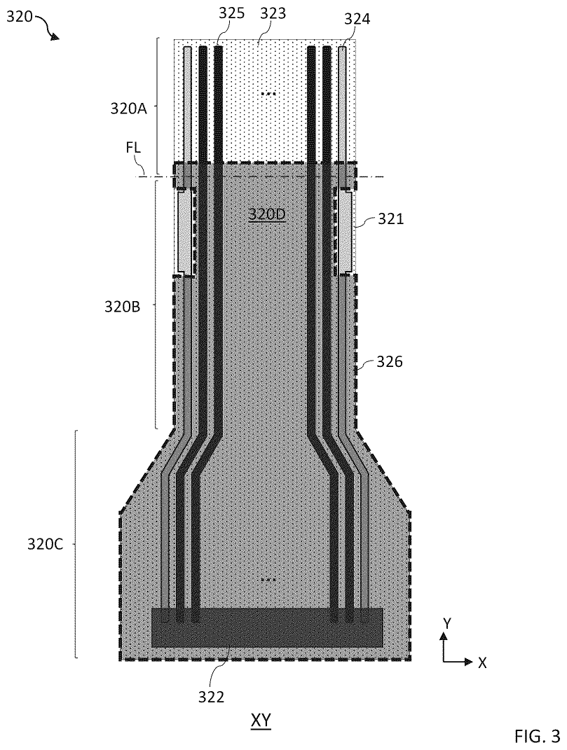

Interconnection substrate 220 comprises conductive interconnection elements (not shown in FIGS. 2A to 2G but shown in FIG. 3 described hereafter), generally in the form of interconnection tracks, or tracks, formed inside and/or on top of a dielectric support, generally flexible. The tracks are generally metallic, for example made of copper. The dielectric support is, for example, made of a polymer material, such as polyimide. Interconnection substrate 220 is, for example, a flexible printed circuit.

The interconnection tracks may be positioned on a single surface of the interconnection substrate. According to a variant, the interconnection tracks may be positioned on both sides of the interconnection substrate. For example, each interconnection track or interconnection track portion on the front surface of the interconnection substrate may extend on the rear surface of the interconnection substrate by means of a conductive via. According to another variant, the interconnection substrate may be a multilayer substrate, such as multilayer flexible substrate, and continue interconnection tracks arranged on the front surface of the interconnection substrate over a plurality of planes, or levels, separated by a dielectric layer, where an interconnection track of one level can be connected to another interconnection track of another level by a conductive via.

Interconnection substrate 220 comprises:
a substantially horizontal portion 220A between piezoelectric layer 211, in particular between lower metal layer 217, and acoustic attenuation layer 215;
a substantially vertical portion 220B coupled to portion 220A and folded against the flank 215A of acoustic attenuation layer 215; and
a portion 220C coupled to portion 220B and which may initially be totally or partly substantially horizontal.

Interconnection substrate 220 comprises, on portion 220B and on its front surface 220D, electrical contact pads 221. The front surface 220D of the interconnection substrate is opposite to rear surface 220E, which faces acoustic attenuation layer 215. Pads 221 may be referred to as ground pads.

Interconnection substrate 220 further comprises, on portion 220C and on its front surface 220D, a connector 222. Connector 222 can be used to connect all or part of the tracks of interconnection substrate 220 to another electrical connection element 30 (shown in FIG. 2G), for example to another interconnection substrate, a rigid or flexible printed circuit, an electronic card, a cable or a cable harness, more generally any element enabling to couple the electrodes 26, 27 of transducer elements 20 (shown in FIG. 2D described hereafter) to electronic circuits external or internal to ultrasonic probe 200 (shown in FIG. 2G described hereafter).

FIG. 2B shows a structure obtained as a result of the insulation of edges of acoustic stack 210. To achieve this insulation, a layer of dielectric material 201 (dielectric layer) is formed on at least some of the edges of acoustic stack 210.

Dielectric layer 201 is not formed on the upper surface 210A of acoustic stack 210.

Dielectric layer 201 is formed at least on the lateral edge 210C (in the XZ plane) of acoustic stack 210, which is the lateral edge positioned above the flank 215A of acoustic attenuation layer 215 (the flank onto which portion 220B of the interconnection substrate 220 is folded).

Depending on the metallization technique implemented in the subsequent metallization step, for example in case of a deposition of a metal layer 202 of “solid plate” type as described hereafter in FIG. 2C, it may be necessary to form dielectric layer 201 on the lateral edge 210D opposite to the lateral edge 210C of acoustic stack 210.

Further, dielectric layer 201 may be formed on one or both transverse edges (in the YZ plane). For example, dielectric layer 201 may surround acoustic stack 210. However, this may not be necessary. Indeed, the transverse edges do not necessarily need to be protected since the two transducer elements 20 which are at both ends of the array can be made inactive, their lower electrodes 27 being coupled, for example, to conductive tracks 324, which are at edges of interconnection substrate 320, as described in more detail hereafter in relation with FIG. 3.

Dielectric layer 201 enables to prevent a short-circuit between the lower electrodes 27 of transducer elements 20 (described in more detail hereafter in relation with FIG. 2D). Indeed, without protection, all the lateral edges of acoustic stack 210 would be metallized, which would amount to at best to decreasing the electric field between the lower electrodes 27 and upper electrodes 26 of transducer elements 20, since this would create side electrodes, particularly on piezoelectric material 211, and at worst to short-circuiting the lower electrode 27 and upper electrode 26.

The dielectric material of dielectric layer 201 may be a resin, for example an epoxy resin, or a silicone, or any other material suitable for polymerization and hardening. The forming of dielectric layer 201 may be performed by means of a “dispenser”, for example a syringe, adapted to depositing the dielectric material in calibrated fashion, which is moved along the edges of the acoustic stack 210 to be protected. As a variant, the forming of dielectric layer 201 may be performed by spraying onto the edges to be protected of acoustic stack 210. These examples of materials, as well as of deposition technique, are not limiting. As a variant, dielectric layer 201 may be formed by a 3D printing technique, using any adapted dielectric material.

FIG. 2C shows a structure obtained as a result of the forming of a metal layer 202 on the structure of FIG. 2B (metallization step), in particular on dielectric layer 201, on top of and around acoustic stack 210, around acoustic attenuation layer 215 along all or part of its height, and on portion 220B of interconnection substrate 220 at least until it is in contact with pads 221.

Metal layer 202 enables to establish an electrical connection between the upper surface 210A of acoustic stack 210 and pads 221, for grounding through upper metal layer 216, which will form the upper electrodes 26 of the transducer elements 20 described hereafter. Metal layer 202 can be referred to as the grounding layer.

As indicated hereabove, this encompasses the case where impedance matching layers 212 and 213 are at least partially conductive, and upper metal layer 216 is on top of impedance matching layer 213. More generally, upper metal layer 216 is positioned differently depending on whether impedance matching layer 212 is conductive or not, and/or impedance matching layer 213 is conductive or not, metal layer 202 being formed to come into contact with upper metal layer 216 whatever its positioning.

Metal layer 202 may comprise a stack of layers, for example a bonding layer on which is positioned a metallization layer. The bonding layer may comprise titanium (Ti), chromium (Cr). The metallization layer may comprise gold (Au), copper (Cu), or aluminum (Al). For example, metal layer 202 is a titanium and gold (Ti/Au) layer. For example, metal layer 202 has a thickness in the range from approximately 200 nm to 200 μm.

The forming of metal layer 202 may be performed by a physical vapor deposition (PVD) technique or any other adapted deposition technique.

FIG. 2D shows a structure obtained as a result of the cutting of acoustic stack 210 along a plurality of YZ transverse planes into a plurality of elements, transducers 20 separated from one another by notches 214, or kerfs. This cutting also includes the cutting of metal layer 202. This cutting also includes the cutting of each of the upper 216 and lower 217 metal layers into, respectively, a plurality of upper 26 and lower 27 electrodes. The cutting, and thus notches 214, preferably extend across interconnection substrate 220 all the way to a cutting plane CP located in backing layer 215. For example, the width of kerfs 214 is in the range from 1 μm to several hundred micrometers, for example equal to approximately 20 μm.

Cutting plane CP is preferably located above the level of pads 221.

Piezoelectric layer 211 is thus divided into a plurality of piezoelectric elements 21. Acoustic impedance matching layer 212 is divided into a plurality of elements 22, and acoustic impedance matching layer 213 is divided into a plurality of elements 23. Each stack of elements 21, 22, and 23 forms a transducer element 20.

Lower electrodes 27 are coupled to, for example are in contact with, some of the tracks of interconnection substrate 220 and form signal electrodes of transducer elements 20. Upper electrodes 26 form ground electrodes of transducer elements 20.

Further, portion 220C of interconnection substrate 220 can then be completely unfolded to be in line with portion 220B, or even deformed to approximate the contour of backing layer 215 and of stiffener 216 (described hereafter).

FIG. 2E shows a structure obtained after optional steps of forming of a mechanical support layer 216, or stiffener, under backing layer 215, and of an electromagnetic shielding layer 203 covering the edges, or flanks, of backing layer 215 along all or part of its height, as well as at least part of interconnection substrate 220. Electromagnetic shielding layer 203 is a conductive layer, for example in the form of a copper adhesive. According to an embodiment, electromagnetic shielding layer 203 covers pads 221. Preferably, electromagnetic shielding layer 203 does not cover connector 222.

FIG. 2F shows a structure obtained at the end of a step of forming of a protective layer 204 which covers and surrounds acoustic stack 210 at least along the height of kerfs 214.

Kerfs 214 are preferably left filled with air and are protected by this layer 204.

Protective layer 204 is not necessarily conductive, the grounding being already ensured by metal layer 202.

Protective layer 204 is, for example, an adhesive polymer film. Protective layer 204 is, for example, an Arclad® brand adhesive polymer film.

Protective layer 204 may be metallized at least on the lower surface, that is, the surface facing metal layer 202.

FIG. 2G shows a structure obtained at the end of a step of forming of a layer 205 made of a material capable of propagating ultrasonic waves, preferably biocompatible, for example a polymer, for example a silicone layer, on the structure of FIG. 2F. The upper surface of protective layer 205 may be planar or form an acoustic lens, for example convex, capable of focusing ultrasonic waves.

This layer 205 may be referred to in the following description as a silicone layer or a silicone lens, without this being restrictive of its shape and material.

Silicone layer 205 covers and surrounds protective layer 204 and may extend vertically up to mechanical support layer 216. Silicone layer 205 may be formed by molding.

Layers 201, 202, 204, and 205 have been shown in transparency to view the elements located underneath.

FIG. 2G illustrates an ultrasonic probe 200 which differs from the ultrasonic probe 100 of FIGS. 1A and 1B mainly in that, in ultrasonic probe 100, conductive film 101 has a dual electromagnetic shielding and grounding function, whereas, in the ultrasonic probe 200 of FIG. 2G, the grounding is ensured by metal layer 202. Protective layer 204 may be metallized to also form an electromagnetic shielding layer, but this is optional.

Further, in ultrasonic probe 100, conductive film 101 is deposited after the cutting of acoustic stack 110, while in ultrasonic probe 200, metal layer 202 is deposited before the cutting of acoustic stack 210, which improves the adhesion of the metal to acoustic stack 210. Then, when protective layer 204, and then silicone layer 205, are deposited, there is less risk of tearing metal layer 202 from the top of acoustic stack 210 and of pads 221, thus decreasing the risk of grounding defects and of a decrease in the performance of ultrasonic probe 200.

FIG. 3 shows an interconnection substrate 320 of an ultrasonic probe according to an embodiment.

For example, interconnection substrate 320 may form or replace the interconnection substrate 220 of the ultrasonic probe 200 of FIG. 2G. Apart from the references of the interconnection substrate, the references of the ultrasonic probe 200 of FIG. 2G are used again hereafter.

Interconnection substrate 320 is shown in a fully unfolded (flat) position.

Interconnection substrate 320 comprises:
a portion 320A intended to be positioned between piezoelectric layer 211, in particular between lower metal layer 217, and acoustic attenuation layer 215;
a portion 320B coupled to portion 320A and intended to be folded against the flank 215A of acoustic attenuation layer 215: this portion 320B supports the electrical contact pads 321 on the front surface 320D of interconnection substrate 320; and
a portion 320C connected to portion 320B in line with portion 320B: this portion 320C supports connector 322 on the front surface 320D of interconnection substrate 320.

A folding line FL has been shown between portion 320A and portion 320B.

Portion 320C, which widens in the X direction relative to portion 320B, has been shown, but this is not limiting and portion 320C could have the same width as portion 320B. More generally, the different portions of interconnection substrate 320 could all have other suitable dimensions.

Interconnection substrate 320 comprises a dielectric support 323 inside and/or on top of which metal tracks 324, 325 extend.

Metal tracks 324 are preferably located near the edges of interconnection substrate 320 and are connected to pads 321. Metal tracks 324 are dedicated to grounding, by being coupled to the upper electrodes 26 of transducer elements 20 via metal layer 202 and pads 221.

Metal tracks 325 are located at the center of interconnection substrate 320, preferably between the two metal tracks 324. Metal tracks 325 are dedicated to the signals from transducer elements 20 and are coupled to the lower electrodes 27 of transducer elements 20. Not all metal tracks 325 have been shown in order to avoid burdening FIG. 3, but there may be more than four (symbolized by the sign “ . . . ”), for example, in the order of one hundred tracks, such as 128 tracks or 256 tracks.

Metal tracks 324 and 325 extend all the way to connector 322, to which they are coupled.

Interconnection substrate 320 is partially covered, on its front surface 320D, by an insulating protective layer 326 (delimited by dotted lines), for example a polymer. Protective layer 326 leaves almost the entire portion 320A exposed so that metal tracks 325 can be coupled to, for example, be in contact with, the lower electrodes 27 of transducer elements 20. Protective layer 326 leaves pads 321 exposed so that grounding layer 201 can contact these pads 321.

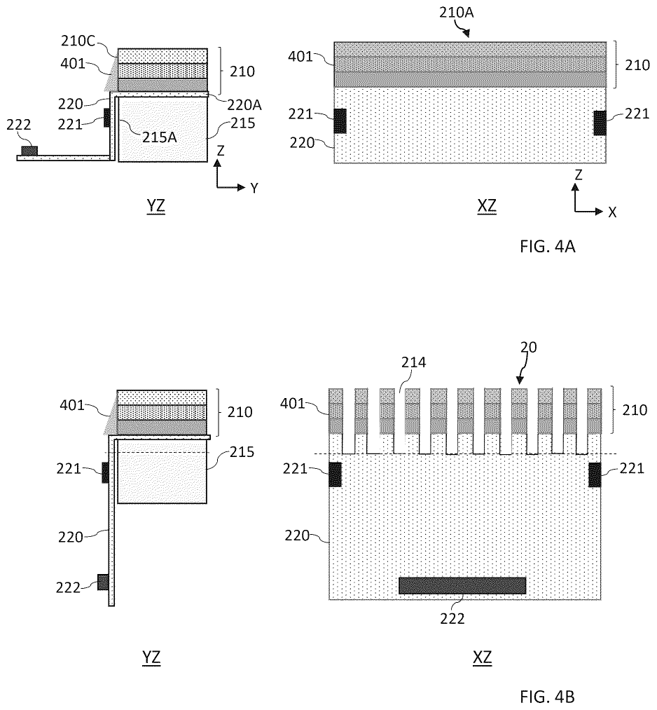

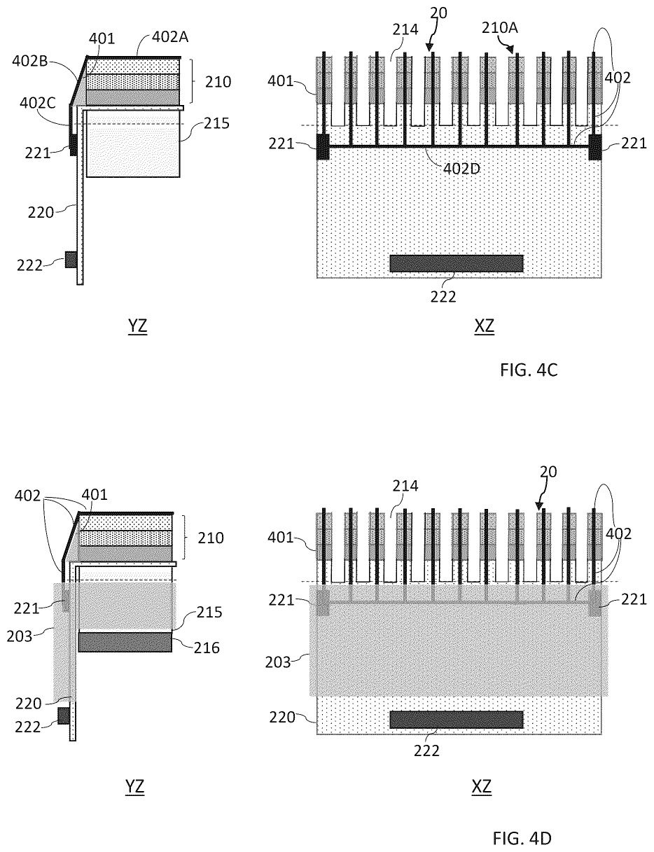

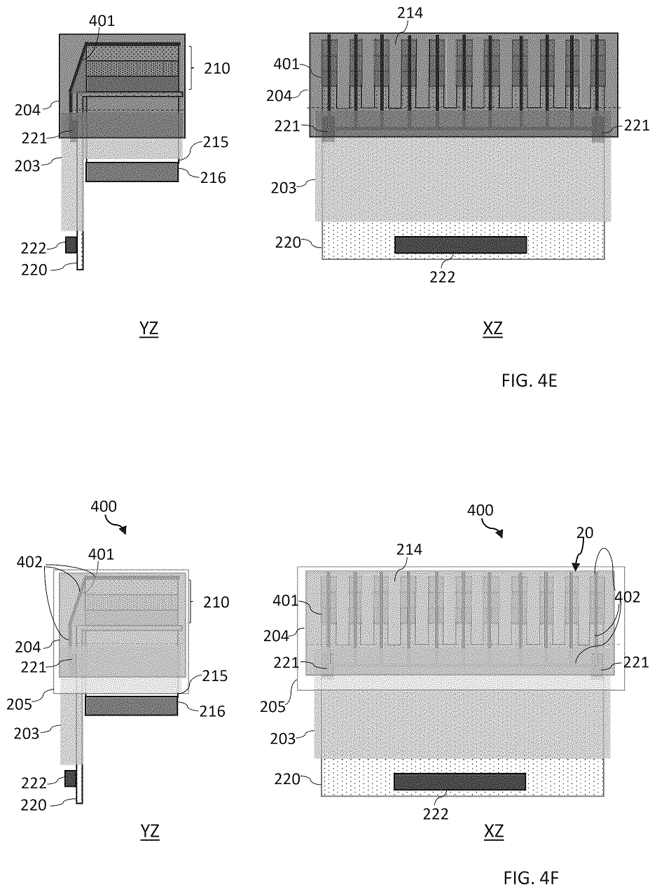

FIG. 4A, FIG. 4B, FIG. 4C, FIG. 4D, FIG. 4E, and FIG. 4F are side and front views illustrating successive steps of a manufacturing method according to another embodiment. The side views are shown in a YZ plane (transverse plane). The front views are shown in an XZ plane (longitudinal or lateral plane).

The manufacturing method of FIGS. 4A to 4F comprises many steps common with the manufacturing method of FIGS. 2A to 2G, which common steps will not be detailed again in the following. Further, the obtained ultrasonic probe 400, shown in FIG. 4F, has many elements in common with the ultrasonic probe 200 in FIG. 2G. These common elements will not be detailed again in the following and will keep the same references.

The manufacturing method of FIGS. 4A to 4F differs from the manufacturing method of FIGS. 2A to 2G mainly in that:
dielectric layer 401 is only formed on the lateral edge 210C of acoustic stack 210, as shown in FIG. 4A;
the cutting of acoustic stack 210 into transducer elements 20 after the deposition of dielectric layer 401 has been performed, but before the metallization step, as shown in FIG. 4B;
instead of a metal layer, the grounding is achieved by forming a network 402 of metal traces on acoustic stack 210 after the cutting thereof, as shown in FIG. 4C.

The network 402 of metal traces comprises upper traces 402A, each formed on one of transducer elements 20, the upper traces 402A are continued by intermediate traces 402B which extend over dielectric layer 401, and the intermediate traces 402B are continued by lower traces 402C which extend at least all the way to pads 221. The two pads 221 are coupled to each other by another metal trace 402D, which also couples the lower traces 402C to each other.

Metal traces 402 may be formed by a 3D printing technique, for example an aerosol jet printing technique, a metal binder jetting technique, or any other technique adapted to forming metal traces.

Instead of a metal layer such as the metal layer 202 of FIG. 2C, the advantage of forming metal traces 402 is that this enables to locally and more precisely achieve the grounding of transducer elements 20, and this, while having fewer edges of acoustic stack 210 to be electrically insulated, in contrast with the insulating layer 201 of FIG. 2B.

Conversely, the embodiment of FIGS. 2B to 2D has the advantage of being easier in terms of process since the metal deposition can be performed over the entire plate, or collectively, rather than being selective. The person skilled in the art may thus select a subtractive-type metallization technique according to FIGS. 2B to 2D, or an additive-type metallization technique according to FIGS. 4A to 4C.

The steps shown in FIGS. 4D, 4E, and 4F substantially correspond to the steps respectively shown in FIGS. 2E, 2F, and 2G, that is:
the forming of mechanical support layer 216 (stiffener) under acoustic attenuation layer 215, and of electromagnetic shielding layer 203;
the forming of protective layer 204; and
the forming of silicone layer 205.

FIG. 4F illustrates an ultrasonic probe 400 which differs from the ultrasonic probe 200 in FIG. 2G mainly in that, in ultrasonic probe 400, the grounding is provided by metal traces 402 instead of metal layer 202. In ultrasonic probe 400, the cutting of acoustic stack 210 is performed before the forming of metal traces 402, but the method of deposition of metal traces 402 enables to obtain a good adhesion of the metal to transducer elements 20, similar to that of ultrasonic probe 200. Then, when protective layer 204 and then silicone layer 205 are deposited, there is less risk of tearing off metal traces 402 from the top of acoustic stack 210 and of pads 221 than for the ultrasonic probe 100 of FIGS. 1A and 1B, thus reducing the risk of grounding defects.

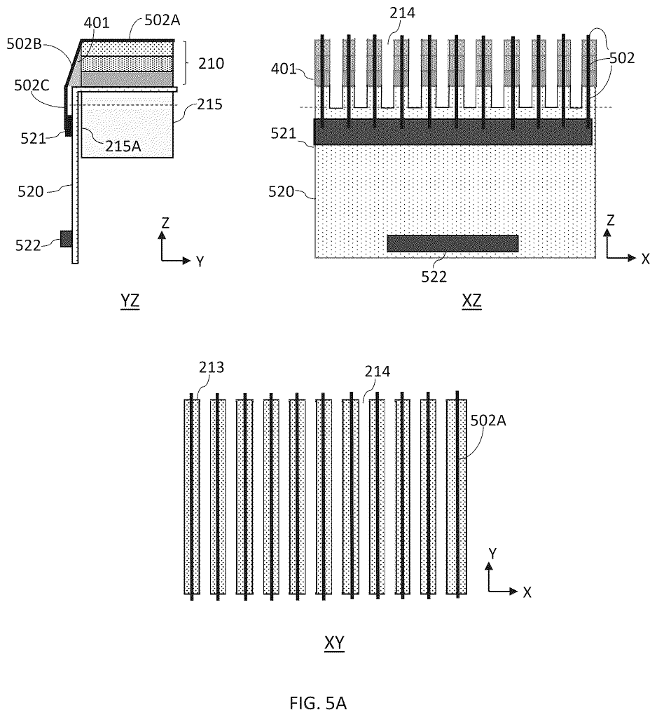

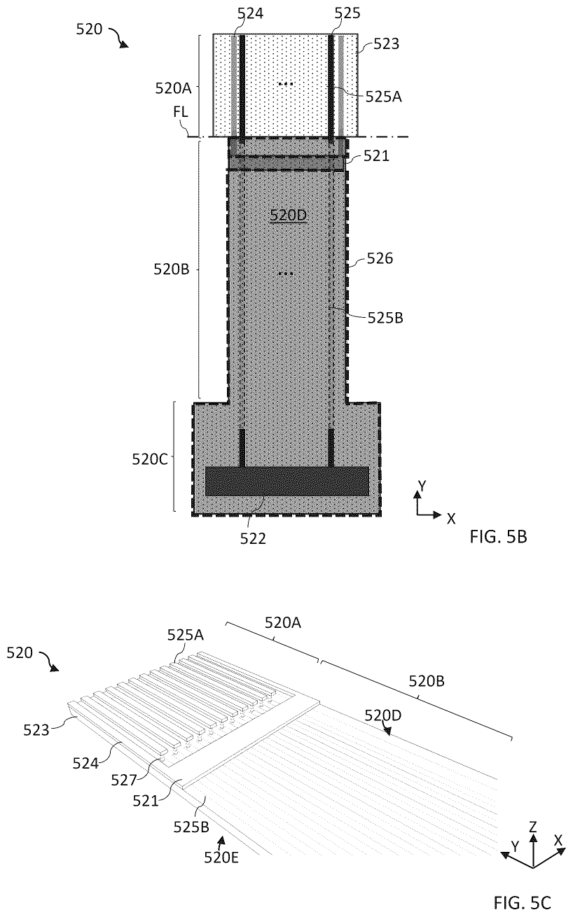

FIG. 5A shows, in side, front, and top views, a variant of the manufacturing method of FIGS. 4A to 4F.

The variant of FIG. 5A comprises many steps common with the manufacturing method of FIGS. 4A to 4F, which common steps will not be described again in the following.

The variant of FIG. 5A differs from the manufacturing method of FIGS. 4A to 4F mainly in that interconnection substrate 520 comprises a single pad 521 instead of two pads 221 for grounding, and in that the network 502 of metal traces comprises upper traces 502A on each of transducer elements 20, intermediate traces 502B on dielectric layer 401, and lower traces 502C which extend all the way to grounding pad 521. It is not necessary for the network 502 of metal traces to comprise the metal trace 402D of FIG. 4C.

The ultrasonic probe (not shown) which will be obtained at the end of the next steps, similar to the steps of FIGS. 4D to 4F, will have the same characteristics.

It should be noted that the top view if FIG. 5A could represent the top view of FIG. 4C.

FIG. 5B and FIG. 5C show an interconnection substrate 520 of an ultrasonic probe according to another embodiment.

For example, interconnection substrate 520 corresponds to, or replaces, the interconnection substrate of FIG. 5A.

Interconnection substrate 520 is shown in a fully unfolded (flat) position. FIG. 5B is a top view, and FIG. 5C is a three-dimensional view, in which dielectric support 523 is shown in transparency so as to be able to view the elements which would otherwise be hidden underneath.

Similarly to the interconnection substrate 320 of FIG. 3, the interconnection substrate 520 of FIGS. 5B and 5C comprises:
a portion 520A intended to be positioned between the piezoelectric layer, in particular between the lower metal layer, and acoustic attenuation layer 215;
a portion 520B coupled to portion 520A and intended to be folded over the flank 215A of acoustic attenuation layer 215: this portion 520B supports electrical contact pad 521 on the front surface 520D of interconnection substrate 520;
a portion 520C coupled to portion 520B and in line with portion 520B: this portion 520C supports connector 522 on the front surface 520D of interconnection substrate 520.

A folding line FL has been shown between portion 520A and portion 520B.

Portion 520C, which is wider in the X direction than portion 520B, has been shown, but this is not limiting and portion 520C could have the same width as portion 520B. More generally, the different portions of interconnection substrate 520 could all have other adapted dimensions.

Similarly to the interconnection substrate 320 of FIG. 3, the interconnection substrate 520 of FIGS. 5B and 5C comprises a dielectric support 523 inside and/or on top of which metal tracks 524, 525 extend. Metal tracks 524 are preferably located near the edges of interconnection substrate 520, and are connected to pad 521. Metal tracks 524 are dedicated to grounding, by being coupled to upper electrodes 26 via metal traces 502 and pad 521. Metal tracks 525 are at the center of interconnection substrate 520, for example between the two metal tracks 524. Metal tracks 525 are dedicated to the signals from transducer elements 20 and are coupled to lower electrodes 27. Metal tracks 524 and 525 extend all the way to connector 522, to which they are coupled.

As shown in FIG. 5C, interconnection substrate 520 comprises at least two interconnection layers, in the example an interconnection layer on front surface 520D and another interconnection layer on rear surface 520E, tracks 525 passing under pad 521.

Thus, tracks 525 comprise tracks 525A on the front surface 520D of interconnection substrate 520 and tracks 525B on the rear surface 520E of interconnection substrate 520, tracks 525A and 525B being coupled to each other in the Y direction by vias 527 between folding line FL and pad 521. At connector 522, tracks 525 can run back to the first surface 520D to be connected to connector 522.

Interconnection substrate 520 is partially covered, at least on its front surface 520D, or even on its rear surface 520E, by an insulating protective layer 526 (delimited by dotted lines), for example a polymer. Protective layer 526 leaves the entire portion 520A exposed so that metal tracks 525 can be coupled to, for example, be in contact with, the lower electrodes 27 of transducer elements 20. Protective layer 526 leaves pad 521 exposed so that metal grounding traces 502 can contact this pad 521.

This embodiment is particularly advantageous because it enables to decrease the width of interconnection substrate 520 as compared with the interconnection substrate 320 of FIG. 3, since pad 521 extends transversely only as far as metal tracks 524.

Various embodiments and variants have been described. The person skilled in the art will understand that certain features of these various embodiments and variants could be combined, and other variants will become apparent to the person skilled in the art.

Finally, the practical implementation of the described embodiments and variants is within the abilities of those skilled in the art based on the functional indications given hereabove.

1. Method of manufacturing an ultrasonic probe, the method comprising:
the forming of a dielectric layer on at least one edge of an acoustic stack comprising at least one piezoelectric layer located on a first portion of an interconnection substrate, said first portion of said interconnection substrate being on an acoustic attenuation layer and being continued by a second portion of the interconnection substrate folded over an edge of the acoustic attenuation layer;
the forming of a conductive grounding structure on transducer elements formed in the acoustic stack and separated by notches resulting from the cutting of the acoustic stack across its entire thickness, said cutting extending at least all the way to the interconnection substrate; said conductive grounding structure extending over the dielectric layer and being coupled to at least one electrical contact pad of the interconnection substrate;
the forming of a protective layer to cover and surround the acoustic stack and the conductive grounding structure at least along the height of the notches.

2. Manufacturing method according to claim 1, wherein the cutting extends into the acoustic attenuation layer and includes the cutting of at least the first portion of the interconnection substrate.

3. Manufacturing method according to claim 1, wherein the dielectric layer is configured to protect the at least one edge of the acoustic stack intended to be covered by the conductive grounding structure.

4. Manufacturing method according to claim 1, wherein the forming of the conductive grounding structure comprises:
the forming of a metal layer on the dielectric layer, on top of and around the acoustic stack at least until it contacts the at least one pad; and
the cutting of the acoustic stack into the transducer elements separated from one another by the notches, said cutting further including the cutting of the metal layer.

5. Manufacturing method according to claim 1, wherein the forming of the conductive grounding structure comprises:
the cutting of the acoustic stack into the transducer elements separated from one another by notches; and then
the forming of a network of metal traces comprising first traces on each of the transducer elements, each first trace being continued by a second trace extending over the dielectric layer and coupled to the at least one pad.

6. Manufacturing method according to claim 5, wherein the second traces are connected to a third trace connected to the at least one pad, or the second traces are directly connected to the at least one pad.

7. Manufacturing method according to claim 1, further comprising the forming of an electromagnetic shielding layer covering the edges of the acoustic attenuation layer along all or part of its height, as well as at least a portion of the interconnection substrate, the electromagnetic shielding layer being a conductive layer, for example in the form of a copper adhesive.

8. . Manufacturing method according to claim 7, wherein the electromagnetic shielding layer also covers the at least one pad.

9. Manufacturing method according to claim 1, further comprising the forming of a layer covering and surrounding the protective layer at least along the height of the notches so as to form a lens, the layer being, for example, a silicone layer.

10. Manufacturing method according to claim 1, wherein the cutting of the acoustic stack includes the cutting of a first metal layer arranged above the piezoelectric layer, for example on the acoustic stack, and of a second metal layer arranged under the piezoelectric layer, for example between the acoustic stack and the acoustic attenuation layer, so as to respectively form first electrodes and second electrodes of the transducer elements, the first electrodes being coupled to the at least one pad via the conductive grounding structure.

11. Manufacturing method according to claim 1, wherein the protective layer:
is a polymer film, for example an adhesive polymer film; and/or
is metallized at least on its surface which faces the conductive grounding structure.

12. Manufacturing method according to claim 1, wherein the electrical contact pad is positioned on the second portion of the interconnection substrate.

13. Manufacturing method according to claim 1, wherein:
the notches are filled with air; and/or
the acoustic stack comprises at least one impedance matching layer on the piezoelectric layer.

14. Ultrasonic probe comprising:
an interconnection substrate comprising at least a first portion and a second portion coupled to the first portion;
an acoustic stack comprising at least one piezoelectric layer located on the first portion of the interconnection substrate;
a dielectric layer on at least one edge of the acoustic stack;
an acoustic attenuation layer, the first portion of the interconnection substrate being between said acoustic attenuation layer and the piezoelectric layer, and the second portion of the interconnection substrate being folded over an edge of said acoustic attenuation layer;
the acoustic stack being divided into a plurality of transducer elements separated by notches extending through said acoustic stack across its entire thickness, said notches extending through at least the first portion of the interconnection substrate and extending all the way into the acoustic attenuation layer;
a conductive grounding structure coupled to each of the transducer elements, the conductive grounding structure extending over the dielectric layer and being coupled to at least one electrical contact pad of the interconnection substrate; and
a protective layer covering and surrounding the acoustic stack and the conductive grounding structure at least along the height of the notches.

15. Ultrasonic probe according to claim 14, further comprising:
an electromagnetic shielding layer covering the edges of the acoustic attenuation layer along all or part of its height, as well as at least a portion of the interconnection substrate; and/or
a layer forming a lens covering and surrounding the protective layer at least along the height of the notches.

16. Ultrasonic probe according to claim 14, wherein the protective layer:
is a polymer film, for example an adhesive polymer film; and/or
is metallized at least on its surface which faces the conductive grounding structure.

17. Ultrasonic probe according to claim 14, wherein the electrical contact pad is positioned on the second portion of the interconnection substrate.

18. Ultrasonic probe according to claim 14, wherein:
the notches are filled with air; and/or
the acoustic stack comprises at least one impedance matching layer on the piezoelectric layer.
